(19) United States
(12) Patent Application Publication
(10) Pub. No.: US 2022/0339030 A1
(43) Pub. Date: Oct. 27, 2022

(54) EYE DROP APPLICATION

(71) Applicant: Bhavesh GOKANI, Hertfordshire (GB)

(72) Inventor: Bhavesh GOKANI, Hertfordshire (GB)

(21) Appl. No.: 17/787,320

(22) PCT Filed: Dec. 19, 2019
(86) PCT No.: PCT/EP2019/086324

§ 371 Date: Jun. 19, 2022

(51) Int. Cl. A61F 9/00

(52) U.S. Cl. CPC A61F 9/0026

(57) ABSTRACT

An eye drop applicator comprises a body (1) of generally inverted bowl shape formed of an opaque material with an opening (3) in the top of the inverted bowl into which the top of a bottle of eye drops can be inserted.

### FIELD OF THE INVENTION

This invention relates to the application of eye drops and has for its object the provision of an improved means for the application of eye drops.

### SUMMARY OF THE INVENTION

According to the present invention there is provided an eye drop applicator which comprises a body of generally inverted bowl shape formed of an opaque material with an opening in the top of the inverted bowl into which the top of a bottle of eye drops can be inserted.

The opening in the top of the inverted bowl is preferably afforded by a tapering formation.

The body may be formed of a resiliently deformable plastic material. If, however, the applicator is intended to be a single use device, it may be formed of paper or from a recycled and disposable pulp material.

The circumference of the body of bowl shape will be such that it can fit comfortably around the eye and the configuration of the body will be such that it can readily be gripped with the thumb and first finger of a hand of a user of the applicator.

There may be a handle projecting from the body of bowl shape.

### BRIEF DESCRIPTION OF THE DRAWINGS

FIG. 1 is a plan view of the eye drop applicator,

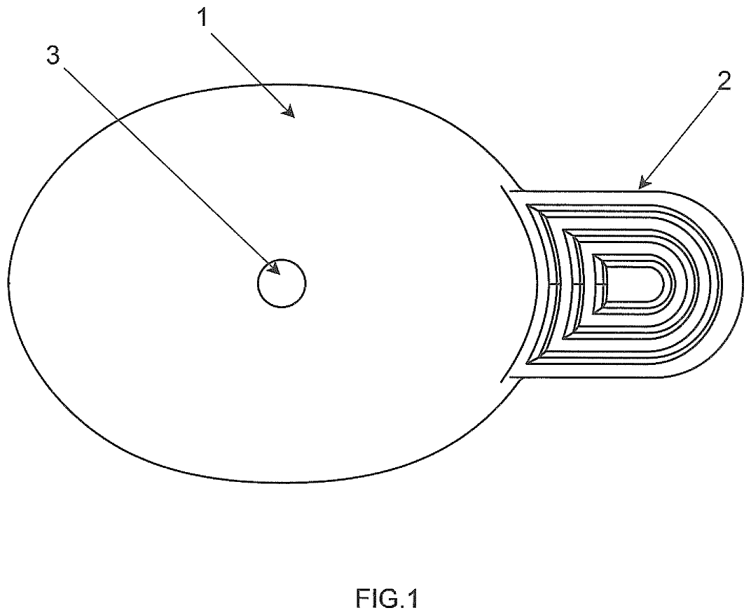

FIG. 2 is a front view of the applicator,

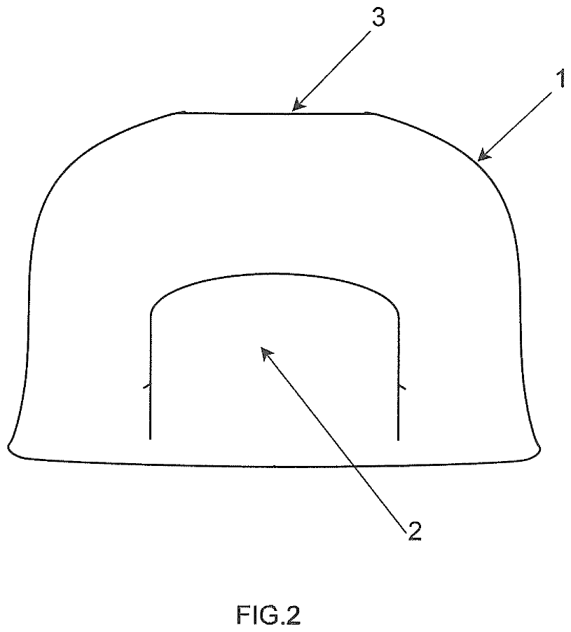

FIG. 3 is a rear view of the applicator,

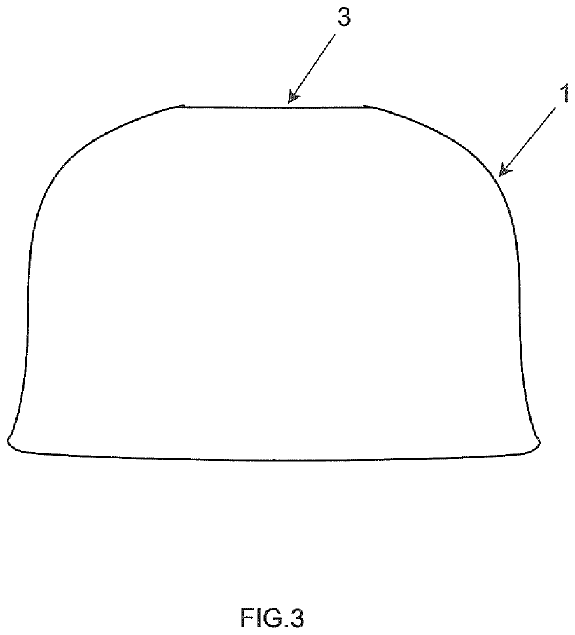

FIG. 4 is a side view of the applicator, and

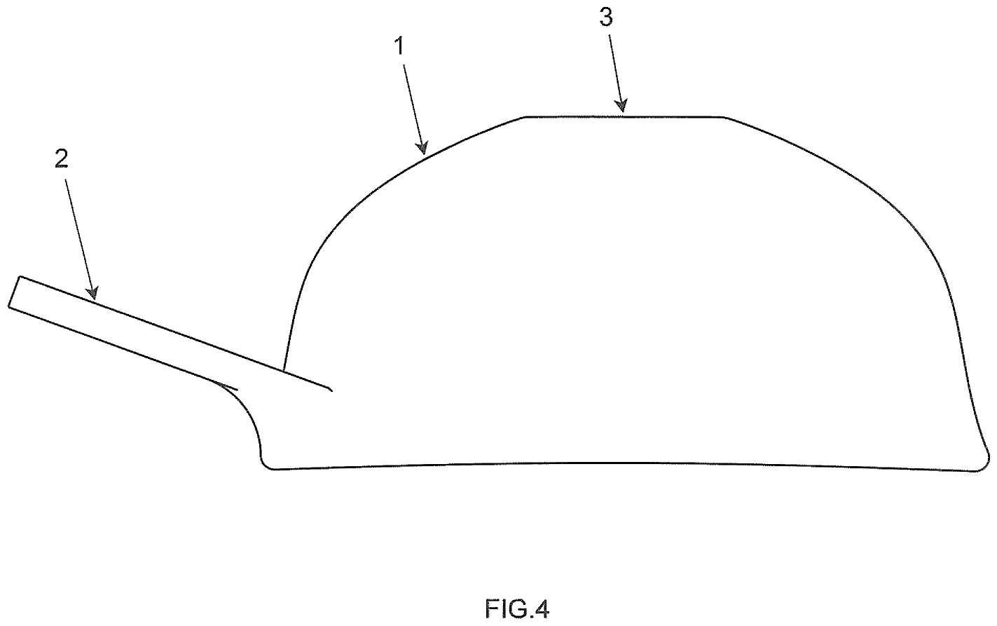

FIG. 5 is a sectional view of the applicator.

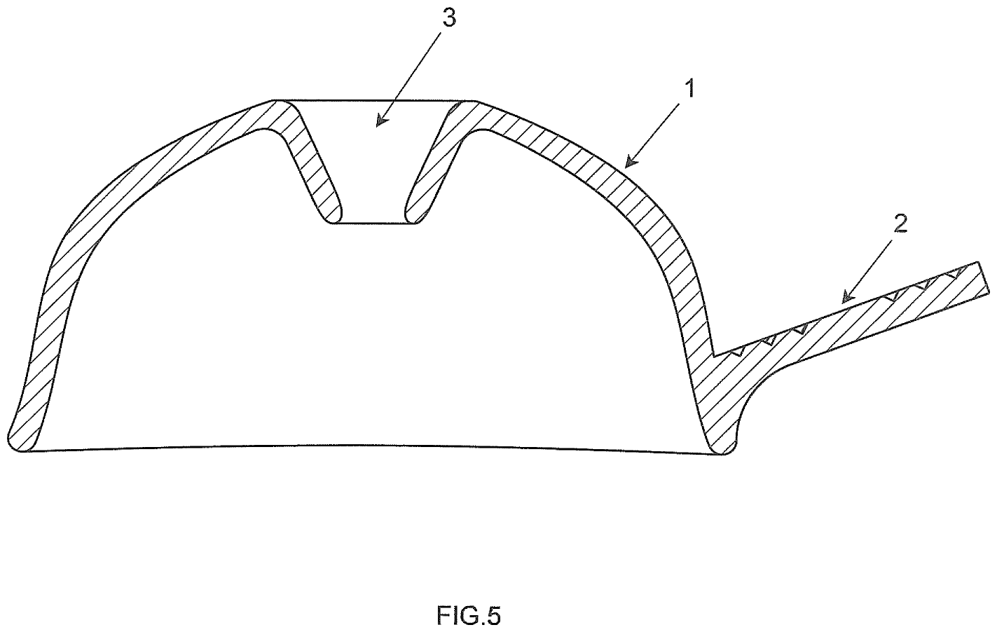

### DESCRIPTION OF THE PREFERRED EMBODIMENTS

The eye drop applicator shown in the drawings is formed from a resiliently deformable plastics material which is either black in colour or a suitable dark shade so that it does not allow any light transmission. It includes a main body 1 having a shape similar to that of an inverted bowl but with an elliptical cross section so that it is easy to grasp in one hand when dispensing an eye drop. There is a central tapering opening 3 at the top of the application and this enables a variety of sizes of eye drop bottles to be inserted into the top of the applicator.

The applicator can be produced by a moulding procedure using a suitable resiliently deformable opaque material.

Before being used, the applicator should first be washed to ensure that it does not carry any dirt. If the user is going to insert an eye drop in his or her own eye, the main body 1 of the applicator is gripped between the first finger and the thumb of one hand with the palm of the hand uppermost. The applicator is then turned over and placed over the eye to be treated and pressed gently into position over the eye so as to ensure that there is no entry of light between the periphery of the applicator and the skin of the user.

With the other hand, the user takes a bottle of eye drops, removes any stopper and places the top of the bottle in the opening 3 at the top of the applicator. The user can see the bottle with vision from the other eye allowing him or her to place the top of the bottle of eye drops accurately and easily in the required position. The eye drop bottle is then squeezed gently so that a drop of the eye drop fluid falls downwardly through the opening 3 and is directed into the centre of the eye. After the eye drop has been applied, the applicator is removed and the user can then use a tissue to wipe the eye and the surrounding area to remove any excess fluid.

The eye drop applicator can alternatively be placed in position using the handle 2, which is grasped between the first finger and thumb, with the thumb on the underside of the handle 2 and the first finger on top of the handle 2. The application procedure is then carried out as described above, with the handle 2 facilitating secure placement of the applicator in the required position.

The provision of the small handle 2 makes it easier for a parent to hold the applicator in place when dispensing an eye drop to a child. It can also be used by a nurse when treating an elderly patient. The upper surface of the handle 2 can be patterned as shown so as to facilitate gripping thereof.

If the applicator is a “single use” device, it can be formed from paper or from a recycled and disposable pulp material with added waterproofing and manufactured in accordance with Medical Devices Directive 93/42/EEC. It will then, of course, not need to be washed before it is used.

1. An eye drop applicator which comprises a body of generally inverted bowl shape formed of an opaque material with an opening in the top of the inverted bowl into which the top of a bottle of eye drops can be inserted.

2. An eye drop applicator as claimed in claim 1, in which the opening in the top of the inverted bowl is afforded by a tapering formation.

3. An eye drop applicator as claimed in claim 1, in which there is a handle projecting from the body of bowl shape.

4. An eye drop applicator as claimed in claim 3, in which the upper surface of the handle is patterned so as to facilitate gripping thereof.

5. An eye drop applicator as claimed in any one of the preceding claims, which is made of a resiliency deformable plastic material.

6. An eye drop applicator as claimed in claim 1, which is made of paper.

7. An eye drop applicator as claimed in claim 1, which is made of recycled material.
